(19) United States
(12) Patent Application Publication
(10) Pub. No.: US 2008/0105393 A1
(43) Pub. Date: May 8, 2008

(54) Process for the Enzymatic Oxidation of a Substrate Comprising a Primary Hydroxyl Group

(75) Inventors: Arie Besemer, Amerongen (NL); Jan Jetten, Zeist (NL)

Correspondence Address: SUGHRUE MION, PLLC, 2100 PENNSYLVANIA AVENUE, N.W., SUITE 800, WASHINGTON, DC, 20037, US

(73) Assignee: NEDERLANDSE ORGANISATIE VOOR TOEGEPAST-NATUURWETEN, Delft (NL)

(21) Appl. No.: 11/573,596

(22) PCT Filed: Aug. 9, 2005
(86) PCT No.: PCT/EP05/08637

§ 371 Date: Dec. 26, 2007

(30) Foreign Application Priority Data
Aug. 11, 2004 (GB) 0417881.0

(51) Int. Cl. C12P 19/04; D21C 5/00; C07H 1/00

(52) U.S. Cl. 162/70; 435/72; 536/123.1

(57) ABSTRACT

The present invention relates to a process for oxidising a hydroxyl group to form an aldehyde and/or carboxy group, the process comprising the step of reacting a substrate comprising at least one primary hydroxy group with a nitrosonium ion in the presence of an oxidase and hydrogen peroxide.

### FIELD OF THE INVENTION

The present invention relates to a process for oxidizing a substrate comprising a primary hydroxyl group, and in particular to a process for oxidizing polysaccharides, and especially for oxidizing primary hydroxyl groups of the cellulose component of cellulosic fibers to aldehyde and/or carboxy groups.

### TECHNICAL BACKGROUND

In recent years much attention has been given by scientists to the selective oxidation of hydroxyl groups of substrates in order to form aldehyde groups and/or carboxyl groups. In the paper industry, such reactions are of significance because these oxidation reactions allow the mechanical properties of paper products to be improved. In particular, the introduction of aldehyde groups into cellulosic material is known to improve the wet strength of paper materials produced therefrom, while the introduction of carboxyl groups is known to improve the dry strength of such paper products. The benefits of oxidized cellulose-containing fibers materials with respect to their non-oxidized counterparts is discussed in WO 00/50462. Furthermore, paper products with desirable characteristics such as high water-absorbability can be produced by further derivatising oxidised cellulose-containing fibers, e.g. by carboxymethylating the oxidised fibers.

It is because of these beneficial properties of the oxidized cellulose materials that so much time and effort has been invested in improving the oxidation processes which yield these superior products. One method of oxidizing primary hydroxyl groups which has proved to be particularly successful is through the use of nitroxy-mediated oxidation reactions. This class of reactions allows the selective introduction of aldehyde and/or carboxylic acids into substrates such as cellulose. The oxidation reaction relies on a nitrosonium ion (A), which is the active species which oxidizes the alcohol to an aldehyde and/or carboxy groups. The nitrosonium ion (A) can be generated catalytically in situ from the corresponding nitroxide (B) in the presence of a primary oxidant as shown in the scheme below. Commonly used primary oxidants include hypochlorous acid and hypochlorite. Depending on the process conditions (e.g. time, pH), the nitrosonium ion (A) will oxidize the alcohol, i.e. the hydroxy compound to the corresponding aldehyde (as shown in the scheme) or carboxy compound while itself being reduced to the hydroxylamine (C). Equimolar amounts of the resulting hydroxylamine (C) and the nitrosonium ion (A) will then recombine (synproportionate) under suitable reaction conditions to form the nitroxy compound (B). Then, the cycle newly starts with the nitroxy compound being oxidised by the primary oxidant to form the nitrosonium ion (A).

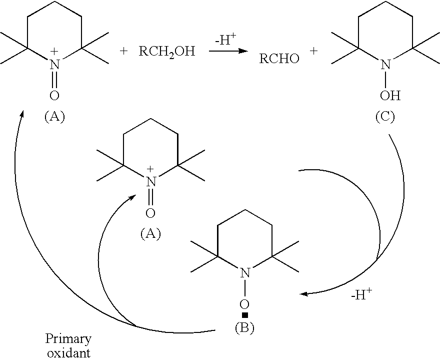

A particularly preferred nitroxy compound for converting primary hydroxy groups to aldehyde groups, is 2,2,6,6-tetramethylpiperidine-N-oxyl which is also known as TEMPO. The use of TEMPO in the oxidation of cellulose and cellulose derivatives is discussed in WO 00/50463.

As will be clear from the above reaction scheme, although the actual oxidation of the alcohol is being carried out by the nitrosonium ion, a primary oxidant is involved which is being spent replenishing the nitrosonium ions from the hydroxylamine derivative. As mentioned above, hypochlorous acid and hypochlorite can be used with good results. However, the use of these agents is not favourable from an environmental point of view, and so recently significant efforts have been undertaken to find an alternative method of forming the active nitosonium ions from the nitroxy compound.

In this regard, WO 00/50463 suggests that this oxidation reaction can be carried out using enzymatic catalysis. Specifically, this document suggests the use of an oxidative enzyme such as a peroxidase or an oxidase. Where the peroxidase is used, hydrogen peroxide is used as the enzymatic substrate. Alternatively, oxidase can be used with an oxygen gas.

However, the methods disclosed in WO 00/50463 are not satisfactory for a number of reasons. Of the two above possibilities for the catalytic oxidation of the hydroxylamine to re-form the active nitrosonium ion, the use of the oxidase is preferred over the use of the peroxidase. This is because only low conversion rates can be achieved when using peroxidase, in part due to the fact that only low hydrogen peroxide concentrations can be used. Furthermore, it is at present much easier to obtain oxidases commercially (i.e. cheaply and in large quantities) than it is for peroxidases.

However, a significant problem when using an oxidase to re-form the nitrosonium ions is that it is most difficult to provide enough oxygen to allow an oxidase/oxygen gas/nitroxy compund system to be efficient. This is mainly because of the poor solubility of oxygen in water, most of paper processing being conducted in aqueous environments. At a pressure of 1 bar oxygen gas, only about 1.20 mM oxygen can be dissolved in water, and when using air it is to be expected that only about 0.24 mM oxygen can be dissolved. Such low oxygen gas quantities will be used up very rapidly in oxidase catalysed systems. Because of the poor solubility of oxygen gas in water, it is difficult to achieve a suitable rate of reaction whereby the nitrosonium ions are re-formed. Furthermore, the use of oxygen gas places severe restrictions on equipment design and further problems arise from the need to stir the reaction mixture in order to help the oxygen dissolve. It would be necessary to use a high-speed stirring system combined with an oxygen dispersion system.

Accordingly, there is still a need for a system for oxidising primary hydroxyl groups of cellulose through the mediation of nitroxy compounds, which is both environmentally friendly and allows a high reaction rate.

The present inventors have set out to develop a process for oxidising hydroxyl groups which shows high chemical selectivity, is environmentally friendly and at the same time allows simple apparatus to be used.

With the deficiencies of the known reaction systems in mind, the present inventors based their work on the previous contributions of nitroxy mediated oxidation systems, and set out to solve the problem of how to provide an alternative oxygen source for the oxidase catalysed oxidation of nitroxy compounds to the corresponding nitrosonium ions.

### BRIEF DESCRIPTION OF THE INVENTION

The above technical problem has been solved by the present inventors by the provision of:

a process for oxidising a hydroxyl group to form an aldehyde and/or carboxy group, the process comprising the step of: reacting a substrate comprising at least one primary hydroxy group with a nitrosonium ion in the presence of an oxidase and hydrogen peroxide.

### DETAILED DESCRIPTION OF THE INVENTION

### Substrate Comprising a Primary Hydroxyl-Group

According to the present invention, there are no specific limitations regarding the nature of the substrate to be used as long as at least one primary hydroxyl group is present. The substrate may represent a clearly defined organic compound comprising a primary hydroxy group, for instance an optionally substituted alkanol. The substrate may also comprise a mixture of compounds with and without primary hydroxyl groups. It must not necessarily be soluble in the reaction medium, but can also be used as a powdery or fibrous insoluble material. In a preferred embodiment the substrate is a carbohydrate. More preferably, the substrate is a polysaccharide. Most preferably cellulosic fibers are treated to oxidise their cellulose content by the presently claimed process. The method of the present invention can for example be used for oxidising carbohydrates of very diverse types and origin (vegetable, animal, microbial, synthetic). Both monomeric carbohydrates (monosaccharides), and dimeric, oligomeric and polymeric carbohydrates, as well as sugar alcohols can be oxidised, if they have a primary alcohol function. Examples of polymeric carbohydrates are β-glucans, such as cellulose (1,4-β), curdlan and scleroglucan (1,3-β) and fractions, derivatives and hydrolysis products thereof, α-glucans, in particular starch (1,4-α) and pullulan (1,6/1,4/1,4-α) and fractions, derivatives and hydrolysis products thereof—such as amylose and amylodextrin-, and cyclic equivalents thereof such as cyclodextrin, also other polysaccharides such as inulin (essentially a 2,1-β-fructan), and natural or artificial gums such as xanthan (1,4-β, with side chains), guar, carob flower, algin, gum Arabic, dragacanth, agar, ghatti, chitin, carrageenin, and the like. In particular, the method is suitable for the oxidation of water-soluble oligosaccharides and polysaccharides such as starch or inulin, or fractions, hydrolysates or derivatives thereof. The use of the claimed methods for the oxidation of cellulose-containing materials such as pulp is particularly preferred.

The pulps which may be used as a substrate for the claimed process may be primary fibrous materials (raw pulps) or secondary fibrous materials, whereby a secondary fibrous material is defined as a fibrous raw material recovered from a recycling process. The primary fibrous materials may relate both to a chemically digested pulp (e.g. Kraft or sulfite pulp) and to mechanical pulp such as thermorefiner mechanical pulp (TMP), chemothermorefiner mechanical pulp (CTMP) or high temperature chemithermomechanical pulp (HTCTMP). Synthetic cellulose-containing fibers can also be used. Preference is nevertheless given to the use of pulp from plant material, particularly wood-forming plants. For example, fibers of softwood (usually originating from conifers), hardwood (usually originating from deciduous trees) or from cotton linters can be used. Fibers from esparto (alfa) grass, bagasse (cereal straw, rice straw, bamboo, hemp), kemp fibers, flax and other woody and cellulosic fiber sources can also be used as raw materials. The corresponding fiber source is chosen in accordance with the desired properties of the end product in a manner known in the art.

The oxidized pulps can be used for the manufacture of paper, in particular tissue paper having improved strength properties. Prior to or after oxidation the starting pulps can be beaten (refined) with the aim of further enhancing paper strength. In the manufacture of oxidized pulps it is further preferred to carry out all oxidation steps in the absence of chlorine-containing oxidants as basis for the production of TCF or ECF paper.

### Cellulose

The substrate may be in the form of cellulosic fibers containing cellulose, the primary hydroxyl groups of which can be oxidized at least in part to form aldehyde and/or carboxy groups. In cellulose, these hydroxy groups are typically at the C(6) position of the glucose-units.

Turning now to define the term “cellulose” for a cellulosic material such as pulp, cellulose is defined here as the long-chain fibrous portion insoluble in 10% (wt. %) NaOH (also known as the R₁₀ portion) and which is also known in older literature as α-cellulose. Instructions as to how to determine the R₁₀ value are given in ASTM Method D1695, Annual Book of ASTM standards, Section 15, Vol. 15.04, American Society for Testing and Materials, Philadelphia 1983 and “Cellulose Chemistry and its Applications”, edited by T. P. Nevell and S. H. Zeronian, Ellis Harwood Pub., West Sussex, England 1985, p. 16 et seq.

The cellulose portion (R₁₀ value) is preferably at least 50 wt. %, e.g. at least 85 wt. %, relative to the total weight of the oven-dried fibrous material. Herein, the term “oven-dried” refers to the determination of the dry content of fibrous material/pulp samples corresponding to DIN EN 20638. If not stated otherwise, all wt.-% and parts by wt. in connection with the cellulosic material refer to the oven-dried material.

Cellulose is present in untreated plant cells, particularly in the cells of lignified plants, in a proportion of up to 50% of the mass, whereas hemicelluloses and lignin account for the remaining 50% of the mass of lignified plants. Depending on the particular variety of plant, cellulose is present in varyingly large proportions (see Dietrich Fengel and Gerd Wegener, Chemistry, Wood, Ultrastructure, Reactions, Walter de Gruyter (1984)). The present invention also covers embodiments wherein the hemicellulose portion is oxidized in addition to the cellulosic chain.

The cellulose in the cellulosic fibers preferably has a an average molecular weight of 50,000 to 2,500,000 g/mol, particularly 50,000 to 500,000 g/mol.

### Nitrosonium Ion and Nitroxy Compound

In addition to its role as an oxidant, the nitrosonium ion is believed to perform a second function in the present reaction system. The present inventors have surprisingly found that the nitrosonium ions derived from the nitroxy compounds can act to decompose hydrogen peroxide to form oxygen gas. The fact that the nitrosonium ions react with the hydrogen peroxide is reflected in the pH of the system which drops when H₂O₂ is added to the system. The reaction scheme is thought to be:

H₂O₂→2H⁺+O₂+2e−2 TEMPO⁺+2OH⁻+2e^{-→}2 TEMPO+2 OH⁻

net reaction:

2 TEMPO⁺+2OH⁻+H₂O₂→2 TEMPO+2 H₂O

Thus the nitrosonium ions may perform a dual function in the present method of oxidizing hydroxyl groups, so that, in principle, no dedicated hydrogen peroxide decomposing agent is needed. However, for such reactions, it is necessary to use a nitroxy compound which is at least partially oxidized so that it is in the form of nitrosonium ions. Dissolved oxygen gas from the atmosphere may be present in small amounts and initiate the reaction.

The nitrosonium ion used in the present invention is derivable from a nitroxy compound. The nitrosonium ion is formed by oxidation of the nitroxy compound.

The nitroxy compound used in the present invention is preferably a sterically hindered organic nitroxy compound. Furthermore, the use of secondary nitroxides is preferred. The presence of one, particularly two bulky groups in the a position to the NO is suitable for steric hindrance of the NO group, e.g. optionally substituted phenyl or aliphatic substituents that are linked to the nitrogen atom of the NO by a quaternary C atom, e.g. tert-butyl. In other words, it is preferred that one, in particular two quaternary carbon atoms are present in α-position of the N-atom. According to a preferred embodiment, at least one, and preferably both α-carbon atoms are dimethyl-substituted. It is similarly preferred that the nitroxy compound from which the nitrosonium ion is derived lacks α-hydrogen atoms. Two substituents can also be combined into an alkylene unit optionally interrupted by a hetero-atom (e.g. O,N) (to form an alicyclic or heterocyclic ring). The molecular weight of the nitroxy compound is preferably less than 500, its carbon number preferably less than 40, in particular less than 30, e.g. less than 20.

Preferred nitroxy compounds can be represented by the following formula (I)

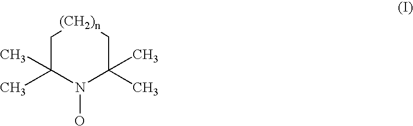

where n=0 or 1 and where the methylene groups of the ring may carry one or more substituents selected from C₁-C₆ alkyl, C₁-C₆ alkoxy, C₅-C₈ aryl, C₅-C₈ aryloxy, amino, amido (e.g. acetamido, 2-bromacetamido and 2-iodacetamido), oxo, cyano, hydroxy, carboxyl, phosphonooxy, maleimido, isothiocyanato, C₁-C₆ alkyloxy, fluorophosphinyloxy (particularly ethoxyfluorophosphinyloxy), substituted or unsubstituted benzoyloxy, e.g. 4-nitrobenzoyloxy. If n=1 (i.e. the ring represents a piperidine), these groups typically substitute the 4-position of the piperidine. Examples are 4-acetamido-TEMPO, 4-acetoxy-TEMPO or 4-hydroxy-TEMPO. The di-tert.-alkyl nitroxy unit can also be present as part of a polymer structure such as {(CH₃)₂—C—(CH₂)₂₋₃-(CH₃)₂—C—NO—}ₘ-. Hydroxy, amino and amido are preferred among these substituents on account of the stability of the nitroxy compound under acidic conditions. The ring may also have one double bond as in 3,4-dehydro-TEMPO.

An example of a compound according to formula (I) wherein n=0 is PROXYL (2,2,5,5-tetramethylpyrrolidine-N-oxyl). DOXYL (4,4-dimethyloxazolidine-N-oxyl) can also be used as nitroxy compound in the present invention.

Preferably, 2,2,6,6-tetramethylpiperidine (TEMPO) is used.

### Oxidase

The oxidase used in the present invention to catalyze the oxidation reaction of the nitroxy compound into the nitrosonium ion is not limited to any particular type of oxidative enzymes. Oxidases are defined as enzymes which catalyze and oxidative reaction using molecular oxygen as their substrate. As specific examples of oxidases which can be used with the present invention, the following can be mentioned: laccases (EC 1.10.3.2), polyphenol oxidases (EC 1.10.3.1) and bilirubin oxidases (EC 1.3.3.5). Polyphenol oxidases (EC 1.10.3.1) include tyrosinases and catechol oxidases such as lignine peroxidase. Suitable polyphenol oxidases may be obtained from fungi, plants or animals. Preferably, the enzyme used in the present invention is a laccase. Laccases (EC 1.10.3.2) are sometimes grouped under the polyphenol oxidases, but they can also be classified as a distinct group, sometimes referred to as p-diphenol oxidase. The laccases can be derived from plant sources or from microbial, especially fungal sources. Specifically, the laccases can be produced by white rot fungi, in particular by the strains Trametes (formerly Coriolus) versicolor or hisuta or villosa. Other known producers of laccase are the strains of the following genera: Agaricus, Armillaria, Aspergillus, Botrytis, Fusarium, Lentinus, Monocillium, Neurospora, Phlebia, Polyporus, Podospora, Pycnoporus and Schizophyllum.

### Hydrogen Peroxide

In order to form oxygen gas (O₂) which, together with the oxidase, oxidizes the nitroxy compound to form the active nitrosonium ion, the present invention makes use of hydrogen peroxide (H₂O₂) which decomposes to form inter alia oxygen gas. The advantage of using such a system to generate oxygen gas rather than feeding oxygen gas into the reaction system is that the hydrogen peroxide can be added in liquid form which avoids the need for dedicated equipment to transfer oxygen gas to the solvent. The use of hydrogen peroxide to form oxygen gas furthermore means that the oxygen gas can be dissolved in the reaction solution more easily allowing e.g. for simple stirring equipment. Furthermore, hydrogen peroxide is already used in many processes in paper-making, for example in bleaching paper so that the process of the present invention can easily be integrated into current paper-making processes and use can be made of already existing equipment.

### Agent which causes Hydrogen Peroxide to Decompose (Optional)

In the present invention, it is not necessary to use an agent which causes the hydrogen peroxide to decompose. This is because the present inventors have surprisingly found that the reaction system can function in an auto-catalytic manner so that the nitrosonium compound causes the decomposition of the hydrogen peroxide as explained above. Accordingly, it is not necessary to use an agent which has this dedicated purpose. However, such an agent may optionally be included, and indeed it is preferable to use a decomposing agent in order to achieve a more efficient production of O₂ from the hydrogen peroxide.

Turning now to address the identity of the optional agent which causes the hydrogen peroxide to decompose, any agent which has such an activity can in principle be used in the present invention. One preferred class of such agents is enzymes which cause the decomposition of hydrogen peroxides, a specific example being catalase. A second preferred class of agents which causes the hydrogen peroxide to decompose is transition metal-containing compounds, in particular compounds of groups 7, 8 or 9 of the periodic table, examples including manganese, cobalt and iron. Of such compounds, salts and oxides are preferred, a specific example being manganese dioxide.

### Reaction Conditions

The process of the present invention can be carried out under a variety of different reaction conditions. As explained above, the substrate comprising a primary hydroxyl group can be employed in various forms. Furthermore, the relative amounts in which a given reagent is used is dependent on the amounts of the other reagents and reaction conditions.

Where the substrate takes the form of cellulosic fibers these may be dispersed in a reaction medium and are preferably present in an amount of 0.1-50 wt. %. A particularly preferable range in which cellulosic fibers are contained is 0.5-20 wt. %, these values being based on the weight of the liquid reaction medium. Even more preferably, the cellulosic fibers are contained in a dispersion in an amount of 1-10 wt. %. The reaction medium can be an aqueous medium, or a homogeneous mixed medium, e.g. of a mixture of water and a secondary or tertiary alcohol or an ether/water mixture, or a heterogeneous medium, e.g. a mixture of water and a water-immiscible organic solvent such as a hydrophobic ether, a hydrocarbon or a halogenated hydrocarbon. In the latter case, the oxidase and/or the nitrosonium ion and the hydrogen peroxide may be present in the aqueous phase and the alcohol substrate and the aldehyde or ketone product may be present in the organic phase. If necessary, a phase transfer catalyst may be used. The reaction medium can also be a solid/liquid mixture, in particular when the nitroxyl is immobilized on a solid carrier. A heterogeneous reaction medium may be advantageous when the substrate or the product is relatively sensitive or when separation of the product from the other reagents may present difficulties.

The nitroxy compound is contained in an amount suitable for the reaction to progress at a desired rate. The amount of nitroxy compound used is usually limited mainly due to the fact that these compounds are expensive. However, it is important not to have too low a concentration of the nitroxy compound as this may limit the reaction rates. Often, quantities of ca. 1 mg/l to 5 g/l of the nitroxy compound will be present with respect to the reaction mixture. If an efficient recovery step is included to allow the nitroxy compound to be re-used or a cheaper source of this component was available, higher concentrations might become practical and could be contemplated. Normally, the nitroxy compound is contained in the reaction mixture in an amount of 0.1-5 parts by weight relative to the weight of the cellulosic fibrous substrate in the reaction mixture where such a substrate is used.

The oxidase is preferably contained in an amount of 120 units/l to 12,000 units/l and preferably in an amount of 240 units/l to 2,400 units/l reaction medium.

Hydrogen peroxide is preferably used in an amount of 0.1 to 10 mmol, more preferably 0.2 to 3 mmol, most preferably 0.5 to 2.4 mmol per 1 reaction medium. Even more preferably the hydrogen peroxide is contained in the reaction mixture in an amount sufficient to maintain an O₂ concentration in the reaction mixture of ca. 1.2 mM per 1 reaction medium. The amount Of O₂ desired, and hence the amount of hydrogen peroxide desired, is dependant also on the reaction conditions and amounts of other reagents. The hydrogen peroxide may be added to the reaction system gradually.

If the optional agent decomposing hydrogen peroxide is a catalase, it is preferably used in amount corresponding to 100 to 5,000 units, more preferably 1,000 to 2,000 units per 1 reaction medium.

A transition metal-containing decomposition agent is preferably used in a molar ratio, based on the molar amount of hydrogen peroxide, of 1/100 to 1/1, more preferably 1/20 to ½, most preferably 1/10 to ¼.

The optional agent which causes the hydrogen peroxide to decompose is most preferably contained in an amount sufficient to decompose the hydrogen peroxide so as to maintain an O₂ concentration of ca. 1.2 mM per 1 reaction medium in the reaction mixture.

The process of the present invention can be carried out under several different reaction conditions. The process can be carried out at a reaction temperature of preferably 0-50° C., more preferably 10-40° C. Furthermore, the process can be carried out at a range of pH values. Preferably, the process is carried out at a pH of 3-7. More preferably, the reaction is carried out at a pH of 5-7. The reaction may be carried out in a closed system which can be pressurised. This allows a higher O₂ concentration to be achieved and avoids the potential problem of O₂ escaping the reaction system.

### Aldehyde and/or Carboxyl Content

The process of the present invention may be used to cause the introduction of aldehyde and/or carboxy groups into cellulosic fibers. The total content of aldehyde and/or carboxy groups in the oxidized cellulosic fibers is preferably more than 50 or more than 100 μmol/g dry weight (oven-dried) of the oxidized cellulosic fibers, particularly more than 150 μmol/g. Even greater preference may be given to values of more than 200 μmol/g, particularly more than 250 μmol/g (e.g. 450 or 500 μmol/g). If the cellulosic glucose units carry a mixture of aldehyde and carboxy groups, the content of C(6)-aldehyde groups preferably also is more than 50, 100, 150, 200 or 250 μmol/g-(e.g. 350 μmol/g or 400) with increasing preference in this order. Preferably the number of aldehyde groups is greater, especially more than two times greater than the number of carboxyl groups.

The oxidized substrates obtainable by the present methods may be subjected to further chemical treatment. The oxidized substrates may be used as starting materials for further functionalisation, especially with alcohols, amines, and other agents capable of coupling with an aldehyde function. Such agents include crosslinking agents (diamines, diols and the like), which can be used to crosslink the cellulose derivatives or to couple them to e.g. amino acids, proteins and active groups. The oxidized substrates may also be coupled with e.g. amines, especially by reductive amination, to produce imino or amino derivatives of cellulose. Also, aldehyde groups of the oxidized derivative can be reacted with hydroxy-functionalized compounds, e.g. glycolic acid, for further derivatisation.

The desirable degree of oxidation depends on the substrate and its application. For various applications, an aldehyde content of about 100 μmol in treated cellulose material is adequate to ensure good wet-strength properties of paper products manufactured therefrom, and higher degrees of oxidation may be disadvantageous. Where the oxidized substrate is to be subjected to further chemical treatment involving the oxidized groups, then it may be beneficial to have a higher degree of oxidation.

For the determination of the C(6)-carboxyl and/or -aldehyde contents in the oxidized cellulosic fibers, or the wet and dry strength parameters of paper test sheets made from the oxidized fibrous material, reference is made to the section “test methods” of WO 00/504

### EXAMPLES

The present invention is now further illustrated by the following examples.

### Example 1

A reaction substrate was prepared by suspending 15 grams of Grapho-Celeste pulp (SCA-Östrand) in 2 l of water. 50 mg TEMPO, 50 mg Laccase (From Trametes versicolor, Wacker Chemie, 12 units/mg), 30 μl of a catalase containing solution having an activity of 100,000 units/ml (NOVO) and 200 μl hydrogen peroxide (30%, Merck) was added to the reaction substrate. The pH was adjusted to 5.8 by the addition of 1 M acetic acid and no further buffer solutions were used. The reaction mixture was heated at 35° C. for 20 hours. During the first hours, a total of 355 μl more hydrogen peroxide was added to the vessel (in increments of 50 μl approximately every 30 minutes).

During the reaction, the following pH changes were observed: first, a gradual increase in pH from approximately 5.3 to 6.0 occurred.

This is believed to be due to oxidation of TEMPO:

4 TEMPO+O₂+2H₂O→4 TEMPO⁺+4OH⁻

Then, the pH remained approximately constant once the pH value had settled at 6.0-6.5. To provide new oxygen gas, hydrogen peroxide was then newly added as set out above. After addition of the hydrogen peroxide, an immediate temporary drop in pH was observed. This is perhaps attributable to the self-decomposition of this system whereby oxidized TEMPO oxidizes hydrogen peroxide:

2 TEMPO⁺+2OH⁻+H₂O₂→2 TEMPO+O₂+2H₂O

After 6-8 hours a gradual pH decrease is observed. This is attributable to the conversion of the hydroxy groups of the pulp to carboxy groups.

The mixture was allowed to react overnight. After one night's reaction, NaOH was added to restore the pH to its original value. Then, the pulp was filtered off and washed several times with water. Then, the aldehyde content was measured by titration with hydroxylamine hydrochloride. It was established that this pulp contained 300 μmol/g of aldehyde groups. From the amount of NaOH needed to neutralize the reaction mixture, it was concluded that approximately 100 μmol/g of carboxy groups had been formed.

### Example 2

Example 1 was repeated, except using 10 mg of manganese dioxide instead of the catalase. All other reagents and conditions were the same.

An oxidized pulp with an aldehyde content of 220 μmol/g and a carboxyl content of 40 μmol/g was obtained.

1. A process for oxidising a hydroxyl group to form an aldehyde and/or carboxy group, the process comprising the step of:
reacting a substrate comprising at least one primary hydroxy group with a nitrosonium ion in the presence of an oxidase and hydrogen peroxide.

2. A process according to claim 1, wherein the substrate is a carbohydrate.

3. A process according to claim 1, wherein the substrate is a polysaccharide.

4. A process according to claim 1, wherein the substrate is in the form of cellulosic fibers.

5. A process according to claim 1, wherein the reaction takes place in the presence of an agent which causes the hydrogen peroxide to decompose.

6. A process according to claim 2, wherein the total content of aldehyde groups in the oxidized substrate is at least 50 μmol/g.

7. A process according to claim 6, wherein the total content of aldehyde groups in the oxidized substrate is at least 100 μmol/g.

8. A process according to claim 1, wherein the nitrosonium ion is derivable from a nitroxy compound represented by the following formula (I): 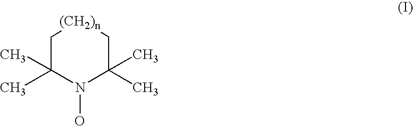
where n=0 or 1 and where the methylene groups of the ring may carry one or more substituents selected from Cᵢ-C₆ alkyl, C₁-C₆ alkoxy, C₅-C₈ aryl, C₅-C₈ aryloxy, amino, amido, oxo, cyano, hydroxy, carboxyl, phosphonooxy, maleimido, isothiocyanato, C₁-C₆ alkyloxy, fluorophosphinyloxy and benzoyloxy.

9. A process according to claim 8, wherein the nitroxy compound is 2,2,6,6-tetramethylpiperidine-N-oxyl.

10. A process according to claim 1, wherein the oxidase is laccase.

11. A process according to claim 5, wherein the agent which causes the hydrogen peroxide to decompose comprises catalase.

12. A process according to claim 5, wherein the agent which causes the hydrogen peroxide to decompose comprises a transition metal compound.

13. A process according to claim 12, wherein the transition metal compound comprises manganese, copper or iron.

14. A process according to claim 1, which is carried out at a pH of 3-7.

15. A process according to claim 1, wherein decomposition of the hydrogen peroxide is achieved through auto-catalysis of the reaction system.

16. A process for oxidising a hydroxyl group to form an aldehyde and/or carboxy group substantially as hereinbefore described.

17. A substrate comprising an aldehyde and/or carboxy group obtained by a process defined in claim 1.
